(19) United States
(12) Patent Application Publication
(10) Pub. No.: US 2006/0135862 A1
(43) Pub. Date: Jun. 22, 2006

(54) Multielectrode array and system for recording and analyzing data or for stimulating tissue

(76) Inventors: John S. Tootle, Vestavia Hills, AL (US); Abidin Yildirim, Birmingham, AL (US); Franklin R. Amthor, Hoover, AL (US)

Correspondence Address: Thomas Kayden;Horstemeyer & Risley, Sutie 1750, 100 Galleria Parkway, Atlanta, GA, 30339, US

(21) Appl. No.: 10/560,474

(22) PCT Filed: Jun. 14, 2004
(86) PCT No.: PCT/US04/18565

§ 371 Date: Dec. 12, 2005

Related U.S. Application Data
(60) Provisional application No. 60/478,054, filed on Jun. 12, 2003.

(51) Int. Cl. A61B 5/04

(52) U.S. Cl. 600/373

(57) ABSTRACT

Disclosed is a multielectrode array suitable for many biological and analytical functions including manipulation of data from neurons, neural prosthetics and biosensing, together with ancillary devices and software for manipulation of data obtained therefrom.

### FIELD OF DISCLOSURE

The present disclosure relates in general to the fields of (1) recording in bodily tissues or other substances for the presence of various phenomena such as, for example, action potentials in neurons or pH in liquids, (2) stimulating bodily tissues with appropriate electrical voltage in the range of millivolts for prosthetics and other applications, and (3) recording and analyzing data received from bodily tissues.

### BACKGROUND

The central nervous system (CNS) and peripheral nervous system (PNS) relate information by means of neurons which transmit electrical activation by means of an action potential through release of neurotransmitters at a chemical synapse or by means of gap junctions in which charged ions flow directly between cells. The patterns of activations of neurons are the language of the CNS and PNS. Electrophysiology is the study of bioelectrical activation of neurons and includes the use of instruments such as the present invention in which recordings are made of neural activation. Recordings of neural activation, as well as other investigative tools, have created knowledge about the workings of the CNS and PNS, although there is obviously much yet to discover.

There has been increasing interest in simultaneous recording of the spiking activity of multiple neurons in the last decade for numerous reasons. One reason is to advance big picture theories of how neurons work together to allow sensory perception, motor activity and other neural activities. Fundamental aspects of neural coding, such as synchronous firing (Singer, 1997), which are not discernable from single cell recordings, have been detected by simultaneous neural recordings. Other hypothesized sensory codes, such as order of firing (Thorpe et al. 2001) also require simultaneous, multi-unit recordings. Multielectrode arrays for recording action potentials of neurons (spikes) are also equally adaptable as means for stimulating sensory (e.g., retina), brain, or other neural tissue.

Multielectrode arrays have universal application within the nervous system, and the retina has been a particularly prominent locus for the development of multi-unit recording arrays. Compared to “brain slice” preparations that are in a depressed state without most of their input, the retina can be removed without cutting the processes of any cells except the axons of the ganglion cells several millimeters from their somas. Since the input to the retina is light, which can be supplied and controlled just as well in the dish as in situ, the retina can be operated in vitro in a nearly normal state of responsiveness for many hours. A technical advantage for array recording is that in retina all the spiking ganglion cells are located in a single, accessible layer close to the surface of the tissue.

There are two basic styles of multielectrode arrays developed for retina recording with respect to whether the ganglion cell layer to be recorded is on the bottom, or top. One of the bottom recording types is that developed by Meister, Baylor and colleagues (1994), and consists of electrodes mounted on the bottom of a chamber in which the retina is placed ganglion cell side down, and stimulated with light from above. Bottom-of-chamber configurations have also been reported by Grumet et al. (2000) and Heuschkel et al. (2002), and exist in commercial versions for brain slice recordings, with typical inter-electrode spacing and construction much different than that of the present invention. Advantages of the dish bottom arrays are that they are rugged, and recordings are obtained merely by placing the appropriate part of the tissue onto the active part of the array. However, most commercially available recording arrays are only marginally suitable for addressing coding issues such as synchronous firing in neural tissue, because few of the neurons, whose inter-soma spacing can be as low as 20 micrometers, will be recorded with electrodes much further apart. The greater density of electrodes in a given area of tissue is a factor in determining whether recording of data gathers the most important characteristics of that tissue. Likewise, greater density of electrodes for stimulating neurons is more likely to approach the kind of density and connectivity in all kinds of neural tissue where, for example, cortical neuron somas can be 20 micrometers apart while connected to more than 5,000 other neurons.

For in vitro experiments, there are also disadvantages to the dish-bottom array approach besides the typical electrode spacing referred to above. The first is that in order to change the location where the array records, the whole piece of tissue must be physically moved. A second problem somewhat particular to retina is that dish-bottom arrays almost always use the isolated retina preparation that is much less robust than the isolated eyecup preparation in which the retina remains attached to the pigment epithelium. Moreover, for ganglion cell recordings, the isolated retina preparation is generally less healthy mounted ganglion cell side down, than up, because superfusion of the ganglion cell side of the tissue promotes the long term health of the spiking cells.

As a recording instrument, the present invention in one embodiment has demonstrated a better than 50% yield that any given electrode will have at least one usable recording, with some electrodes yielding 2 or 3 usable ganglion cell recordings, so that overall, nearly as many ganglion cells can be recorded as array elements. Retinal recordings are stable for 4-6 hours or more, and different regions of the retina can easily be investigated by moving the array to a new retinal position. The invention also has the advantage of being usable for long periods of time, and is easily fabricated by hand using routine technology likely to be found in any electrophysiology laboratory. The invention is uniquely suited to over-sampling a given area of the neural tissue so that there is a high probability of recording simultaneously from many of the neurons in a given area of neural tissue. Likewise, the invention's small distances between electrodes allow simultaneous stimulation of many of the neurons in a given area of neural tissue.

Multi-array configurations that record from the retina ganglion cell side up have also been developed by Normann and coworkers (1996) and Sandison et al. (2002), among others. Recording from above permits easy and rapid movement of the array to an optimum location in the tissue based on the results obtained. However, in the retina, problems with some of the extant superior-approaching arrays include inter-element spacing that is too large, or inability to use the intact retina-pigment epithelium mounting because the array is not transparent, forcing visual stimulation to come from the side of the tissue opposite from the electrode. In retina, a relatively transparent multi-electrode array, one of the embodiments described herein, allows recording in either the eyecup or isolated retina preparation.

### BRIEF DESCRIPTION OF THE FIGURES

FIG. 1A is a diagram of a portion of one layer of the multi-electrode recording array. The mica or acetate substrate is glued to the electrical connector with epoxy. Individually made carbon fiber electrodes are laid in each layer by stabilizing the electrode tips in a fine mesh mounted below the recording end of the substrate (not shown), which is removed after the assembly is complete. The insulated carbon fibers are bonded to fine copper wires, which in turn are soldered to the electrical connector. Polyurethane insulates the individual carbon fibers, and bonds the assembly together.

FIG. 1B is a diagram of the side view of the multielectrode recording array showing how layers are offset with respect to each other, so that when advanced at a 45 degree angle, all electrode tips are in the same horizontal plane. This permits visualization of the underlying tissue through the electrode tips to the substrate, and through the substrate farther from the tips.

FIG. 2A is a copy of a photomicrograph of the tip region of the multielectrode recording array's single insulated and silver-plated carbon microelectrode. The shiny area is the exposed, plated region. The scale is 100 microns.

FIG. 2B is a copy of a photomicrograph of a two layer, ten electrode multielectrode recording array placed over a graticule whose large, labeled divisions are 100 microns apart. The view is from above, through the microscope objective, which can also be the stimulus path (although stimulation can come from below if an isolated retina, rather than eyecup is used.) The graticule is clear visible beyond the substrate for approximately 50 microns. The graticule scale is also visible through the substrate at the upper left, although the acetate substrate refraction shifts the image. The refractive shift can be minimized by using thinner mica rather than acetate as the substrate. The scale is 100 microns.

FIG. 3 is a schematic diagram of the electronic amplifier used for each channel, with the gain versus frequency plot below. Resistances are in megaohms (M), capacitors in picofarads (p), and frequency is in log units (log scale). All operational amplifiers are T1081 equivalent (either T1082 dual or T1084 quad versions are actually used).

FIG. 4 contains traces from simultaneous recordings from the ten electrode multi-array shown in FIG. 2A and FIG. 2B. Channels 4-8 have easily discernable spikes even at this low resolution scale. A signal-to-noise index was measured as the ratio of the peak to peak spike height to the peak to peak noise 2 ms away for 100 spikes for each channel. These values for channels 0-9 are, respectively: 4.3, 4.8, 4.7, 3.2, 9.2, 7.9, 7.7, 6.1, 4.9, and 4.0.

FIG. 5 shows use of template cross-correlation to distinguish spikes of similar amplitude, but different shape. (A) Two portions of the raw trace showing templates derived from spikes with different shapes. (B) Normalized cross-correlation plot showing the separability of these different units by different cross-correlations with the two templates.

FIG. 6 contains PST histograms of the eight units derived from the ten element multi-array shown in FIG. 2B. In this case, no more than one unit was derived from each channel, but, depending on the tip size and location in the retina, sometimes 2 or 3 distinct units are derivable from single channels using the template method. Case 9 shows the response of a unit to a left-to-right sweep of a bright bar, case 10 to a right-to-left sweep.

FIG. 7 is a drawing of an idealized cross-section of neural tissue (cross-hatched) showing only the somas of three neurons which are drawn to represent different layers of the neural tissue as in, for example, layers 1-6 of mammalian neo-cortex. The substrate of the prosthetic device rests on the surface of the neural tissue, and individual electrodes protrude at different distances from the substrate into the neural tissue. Four electrodes are represented, one is a stimulating electrode, and another is a receiving electrode. Two other electrodes illustrate a sharpened tip of an electrode, and the exposed end of the electrode which protrudes beyond the substrate of the array.

FIG. 8. is a flow chart showing the elements of the feedback loop.

FIG. 9. is a flow chart showing the elements of the biosensing electrode circuit.

### DETAILED DESCRIPTION OF THE PREFERRED EMBODIMENTS

I. Multielectrode Recording Array for Retina

As a recording device, the multi-electrode array system consists of three main elements: (1) the array and its mounting, (2) a data collection system for the recordings, and (3) a data extraction, stimulus control and analysis system.

Described is a multielectrode array with metal or carbon fibers which can be used either to record various phenomena such as action potentials. If desired, the tips of carbon fibers from the substrate can be coated with metals such as gold or silver to increase conductance of electricity. Also, if metal wires are used instead of carbon fibers, the exposed ends of these metal wires can be coated with a more biocompatible substance such as carbon. Although what is described is the embodiment for recording in the retina, the multielectrode recording array and system can be used to record from any neural tissue.

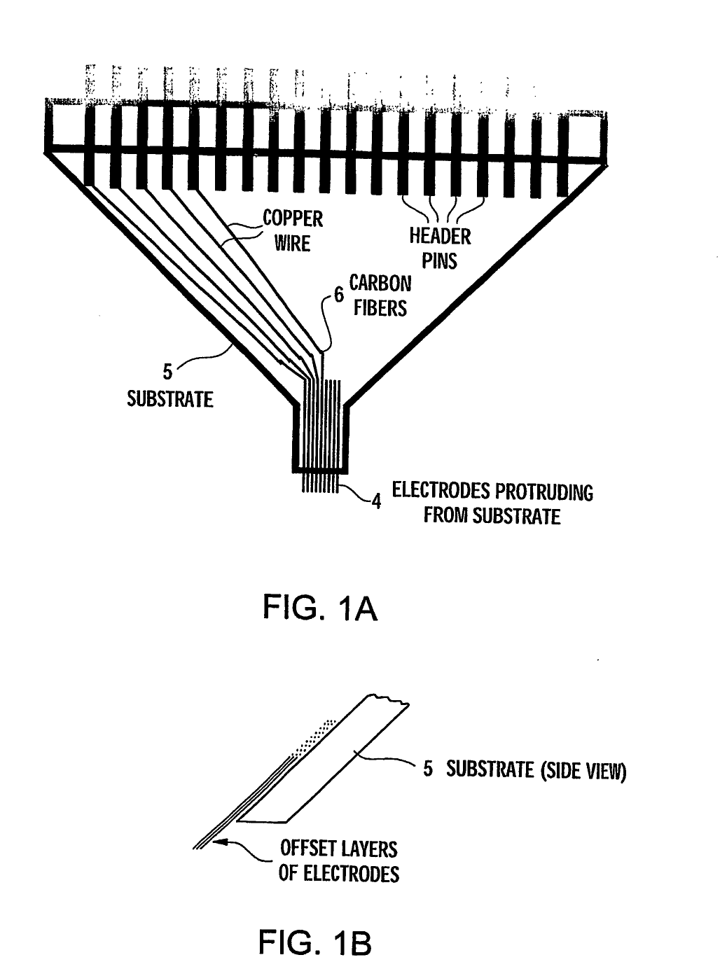

The retinal array and its mounting. The array consists of a set of individual microelectrodes mounted on a substrate that, in turn, is attached to a standard, high density 0.050 inch grid electrical connector called a “header” or “interconnect” (Mill-Max, Oyster Bay, N.Y.) that is available in single and multiple row versions of various number of pins. The substrate, which can be transparent (for example, mica or clear acetate) is glued to this header, and the electrodes are bonded to the substrate. The clear substrate has a roughly triangular shape, being wide at the connector end, and ending in a several hundred micrometer wide tip region from whence the electrodes will emerge, as shown schematically in FIG. 1A. The individual electrodes are typically 8 or 12 micrometer diameter carbon fibers (Thornel T-500, 12K, Amoco Performance Products, Greenville, S.C.) insulated with polyurethane, although tungsten wire (1 mil, 25 micrometer diameter) has been used as well.

The multielectrode array is made from a number of individual electrodes, and each electrode is suitable for recording, and can be tested for such. The carbon fibers typically are supplied in loose bundles. Lengths of fibers are cut off the end of one of these bundles and the ends of a number of the fibers are lightly pressed onto the edge of a piece of double-sided (double-stick; Scotch) cellophane tape. The fibers are “fanned out” under a dissecting microscope so that some of the individual fiber ends are separated. A three centimeter length of thin bare copper wire (#43AWG; 50 micrometers diameter) is advanced by a micromanipulator until the copper wire overlaps an individual carbon fiber by about 1 mm. A small drop of conductive bonding agent such as colloidal silver paste (Ted Pella, Fadding, Calif.) is used to bond the carbon fiber electrically and mechanically to the copper wire. The wire-fiber assembly is then pulled off the tape, and the overlap region is coated with a clear bonding agent such as polyurethane (Delta Ceramcoat Gloss Exterior/Interior Varnish, Whittier, Calif.) for additional mechanical integrity and insulation.

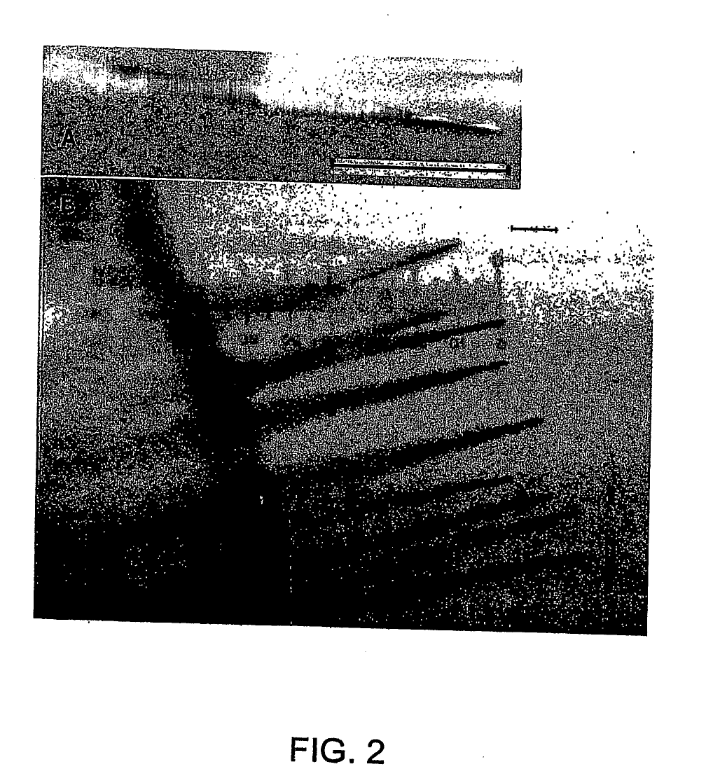

The fibers are then electrolytically sharpened by bringing a high voltage (1000-2000 volts DC) positively charged stainless steel point up to the carbon fiber as the negative pole, until a single small spark erodes away a length at the tip of a few tens of micrometers. The carbon fiber is then insulated to within about 10 micrometers of the tip by lowering it into a small drop of polyurethane in a “U” shaped piece stainless steel wire, and advancing and retracting the fiber repeatedly (the tip itself never enters the polyurethane) until about 10-20 coats of polyurethane are made from the tip region to the end at the overlap with the copper wire, already insulated with polyurethane. This is done under a dissecting microscope to insure that the tip of the carbon fiber never enters the polyurethane drop. One of the principal reasons for choosing polyurethane as an insulating material was its surface tension and slow drying property made it possible to insulate the electrodes up to the tip in one step as above. The last step in making the individual microelectrode is to silver-plate the tip region by putting the tip in a drop of silver electroplating solution (Vigor Silver Electroplating Solution, B. Jadow & Sons, New York, N.Y. 10010) and passing a few microamps of current (tip negative) for a few seconds under dissecting microscope observation. An individual electrode can be made in a few minutes. The tip end of a polyurethane-insulated carbon fiber is shown in FIG. 2A.

The impedance of the electrodes can be determined during the silver-plating procedure. Electrodes that have insufficient exposed tip do not pass enough plating current and do not record well. This can be correlated with the actual impedance. Because the plating can be done before the assembly, these can be rejected from use. The impedance of the electrodes in the array is measured with a constant current of 100 nA at 1000 Hz. Most electrodes have impedances between 500 kΩ and 1 MΩ. A few electrodes have impedances between 1 and 5 MΩ and record less reliably than those with lower impedances. Electrodes with impedances higher than 5 MΩs rarely yield usable recordings.

Instead of carbon fibers, wires composed of biocompatible metals such as platinum, titanium, iridium, a platinum-iridium alloy, silver or other similar metals can be used. Use of these biocompatible wires would eliminate the need for a coupling between the carbon fibers and the metal wire embedded in the substrate.

In order for the array to be transparent for working in the retina (with only the carbon fibers obstructing a small percentage of the light path), it is necessary to remove the copper wire portion of the electrodes from the optical stimulus path. This is done by mounting the electrodes on any transparent substrate (for example, mica or clear acetate) that can be advanced toward the retina at a 45 degree angle. Because retinal ganglion cells to be recorded lie in a single layer, this in turn means that the tips of the electrodes must be precisely placed so that when advancing along the axis of the electrodes, all tips lie in the same horizontal plane. The offset compensation for this angular approach is shown in FIG. 1B.

The individual microelectrodes are placed in the array in layers. Although the numbers can be varied for any purpose, there are typically 6 to 10 electrodes per layer. For the alignment, a small piece of nylon mesh (Monofilament Cloth, 20 microns, Small Parts, Miami Lakes, Fla. 33014) should be attached temporarily at the tip of the substrate on which the electrodes will be bonded at the angle at which the retinal surface will be encountered when the electrode array is advanced into it. Just below (about 25 micrometers) this mesh is an acetate layer that the electrode tips will rest against during assembly. The individual microelectrodes are lowered by a micromanipulator so that the carbon fiber goes through the mesh, and rests against the clear acetate. A small drop of polyurethane bonds the carbon fiber in place near its exit from the substrate. When this first drop has set for a few seconds, a second drop bonds the copper wire to the substrate, and the other end of the copper wire can then be released. Then the next microelectrode is laid in that layer, and the next, until the layer is finished. At this point, for additional layers, either a few coats of polyurethane are added over the entire first layer, or a small piece of mica is bonded with polyurethane to the top of the first layer, as the substrate for the second layer. Finally, after all the individual microelectrodes are assembled, the copper wires are soldered to the pin connectors of the interconnect-header.

Because carbon fibers are so flexible, the tips extend beyond the substrate less than about 1 mm, otherwise, surface tension in the superfusion fluid can cause the tips to bend towards each other and in some cases stick together. The big advantages of the millimeter gap between the electrode tips and the substrate, however, are that only the tips are in the retina, minimally disrupting it, and unimpeded flow of superfusate over the tissue. In addition, in the retina, microelectrodes that do not extend enough beyond the substrate tend only to record from axons, resulting in receptive fields that are not distributed tightly around the site of the array, as needed for examining population coding. FIG. 2B shows an array with two layers of electrodes whose centers are 150-200 microns. The polyurethane coating on the 12 micrometer carbon fibers brings the total diameter of the insulated fiber to less than 20 micrometers, so that inter-electrode spacing is less than 20 microns if they are packed together. Use of smaller diameter carbon fibers (e.g. 4 microns) could produce electrodes whose spacing of centers would be less than 10 microns. Current array electrodes in the prior art are typically on much larger centers. Electrodes have some variability in the electrode spacing. The electrode tips may be off-center by as much as ±40 μm, and the tip extension by ±25 μm.

A retinal array of 16 elements can be constructed in about 12 hours working time at a station where all the supplies and jigs remain setup. The construction of each individual microelectrodes takes less than 10 minutes, which includes bonding the carbon fiber to the copper wire, etching the carbon fiber to a point, coating the carbon fiber up to the tip with polyurethane, and then silver-plating the tip. The longest time is used in adding each electrode to the assembly. The finished microelectrode is lowered until the carbon fiber lies on the assembly, and then advanced until the carbon fiber tip passes through the correct hole in the mesh. This sometimes takes several attempts. The most difficult part is placing a very small drop of polyurethane on the fiber near the edge of the array to make the initial bond. Then a larger polyurethane drop is applied farther up the fiber, and another over the copper wire. This process can take 20-30 minutes per electrode. The final process of soldering the free copper wire ends to the connector takes only about 15 minutes, using a very small tipped batter soldering iron. Because the substrate for the array is glued to the side of the connector, the contact points of the connector for soldering are off the surface of the substrate and do not get contaminated by the polyurethane. Neither does the low temperature soldering affect the substrate.

Mounting and manipulation of the retinal array embodiment. The array is plugged into a female connector that is attached to the array amplifier. This connector is mounted on a standard micromanipulator like that for a single electrode, but with the following difference: the manipulator has been equipped with rotation axes that allow the array to be rotated in pitch and yaw, in addition to the conventional axial movement. This is because, in the retina, the plane of microelectrode tips should be even and parallel to the retinal surface to record simultaneously from a single layer of ganglion cells. Slight rolls in the tissue require tilting the array to achieve this, since the individual microelectrodes are not moveable relative to each other. Once properly oriented, the array is used to explore different regions of the retina or other tissue in a manner similar to that used by a single electrode. Retinal ganglion cells, stained with Azure B (Amthor and Oyster, 1995) can be seen through the array, as the only portion of the field that is blocked is the small percentage due to the carbon fibers in the field of the objective lens (5×, Nikon), as shown in FIG. 2B. Stimuli in the system for use in the retina can be delivered through the array using the epi-illumination pathway via a 100% reflecting cube (XF125, Omega Optical, Brattleboro, Vt.). For monitoring the stimulus location, a 50% reflecting “metallurgical” cube (Nikon) can be used to view the retina, array, and stimulus spot simultaneously.

Retinal recording and data acquisition embodiment. Although low noise, high input impedance amplifiers of up to four independent channels are relatively commonly available (such as A-M Systems), units with 16 or more channels are harder to find, and are much more expensive. The present invention includes a simple, inexpensive amplifier system based on the popular, high input impedance TL081 op amp that allows placement of a large number of channels in a small box at very low cost. Some of its overall features contributed significantly to the efficiency and success of the system which can be constructed with commonly available materials. For example, the TL081 operational amplifiers have been available for more than 20 years, and now exist in dual and quad miniature surface mount versions, which allow considerable savings in space and costs. The space saving allowed construction of small 4×4 inch boards with 16 complete channel amplifiers on each board, which in turn allowed mounting of all the electronics within a few inches of the array.

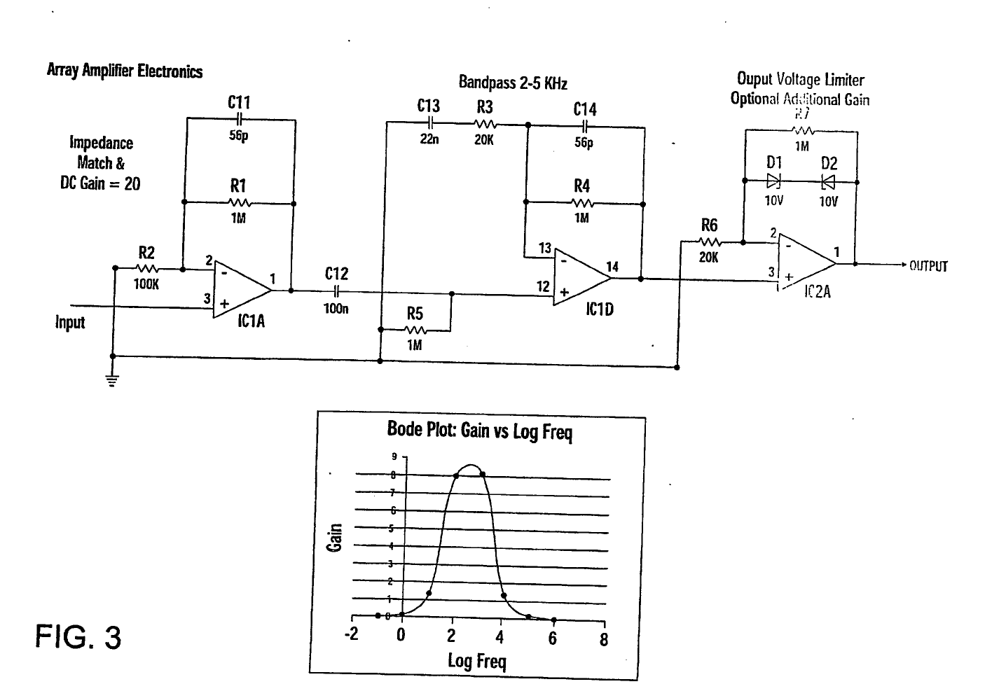

Each amplifier consists of three stages: a preamp, an active bandpass filter, and an output voltage limiter with optional gain and notch filtering. The schematic of the amplifier is shown in FIG. 3. The preamp is DC-coupled follower-with-gain, with a fixed gain of 10, and a high frequency roll-off feedback capacitor. The DC coupling takes advantage of the high input impedance of the opamp. The preamp gain is limited to 10 because the use of dissimilar electrode types and coating sometimes produces offset junction potentials that can saturate the amplifiers at higher DC gains. The middle stage is a bandpass filter with a center frequency of about 2.5 kHz, as shown in the Bode plot in FIG. 3. The lower limit of the bandpass filter limits the intrusion of 60 Hz line noise, and other slow potentials such as due to movement of the superfusion fluid. The high limit excludes signals outside those produced by neurons, and avoids aliasing the A/D converter.

The last stage is optional, and is primarily related to the particular A/D board used (i.e., Measurement Computing Corp., formerly known as Computer Boards) which operate on maximal ±10 volt input ranges. If the power supply for the amplifier is greater than ±10 volts, then the A/D inputs need to be protected. Additional gain can be added to boost the signal further to “fill up” the A/D converter, which was more of an issue with a 12 bit than a current 16 bit A/D converter. The system also contains circuitry in some versions of this amplifier to further exclude 60 Hz line noise, but careful control of electrode impedance and ground loops eliminated most of this noise without this portion of the circuit. Thus, proper choice of power supply and A/D converter can make this stage unnecessary. If so, then, one can record with circuitry with only two opamps, 5 resistors, and 4 capacitors per electrode channel.

Acquisition and Control of Retinal Data Embodiment. Although the array is manipulated much like single electrodes, recording with arrays differs from single electrode recording in one particularly significant aspect. With a single microelectrode, one changes the position and depth of the electrode until an adequate signal to noise ratio is obtained so that spike times can be acquired with a threshold device such as a Schmitt trigger. But with an array, since one cannot move the microelectrodes individually, there will be some proportion of the channels that have poor signal to noise, or multiple spikes of similar heights. At least in experiments in rabbit retina, acquiring all the data the array was capable of producing demanded analog data acquisition and post-acquisition processing.

Data Acquisition. Adequate computer power and analog to digital conversion cards have become very inexpensive recently: 64 channel 16 bit A/D cards and the computers to house them are within the purchasing power of most laboratories, although the expertise to program them may not necessarily be. The system for generating impulses for testing and acquiring data runs on Microsoft Windows-based computers using 64 channel (single ended) A/D boards from Measurement Computing Corp. Stimulus generation, all software for data acquisition, and analysis was developed using Microsoft Visual Studio 6.0 and the Measurement Computing Universal Library. Direct programmatic access to video hardware, necessary for high quality graphical animations of the stimulus display, was accomplished using Microsoft DirectX 7.0.

Data acquisition and stimulus presentation were synchronized to the vertical retrace of the display monitor (100 frames/s). In recordings of the output of a photocell placed on the display monitor, the variability in the interval between the start of data acquisition and the time that the stimulus appeared at the photocell's position on the display was in a range of observed intervals ≦1 ms for 500 consecutive stimulus presentations.

In the embodiment of the present invention for retinal recordings there are 3 primary computational needs: (1) acquiring the analog data from 16 or more electrodes at rates up to 10 kHz per channel, with minimal delays for storing and processing these data, (2) coordinating this data acquisition with the presentation of complex visual stimuli, which was also computer resource intensive, and (3) monitoring the performance of the array with a software-implemented “virtual oscilloscope” and giving feedback to the experimenter about the cells spike responses, such as post stimulus time histograms.

There are various ways of parceling out these three tasks, such as doing them all on a single fast computer, or dividing the tasks among several computers. One method presumes that the large demands of data acquisition would tie up one computer completely, so that a second computer, hand shaking with the first, would do stimulus generation and analysis. Although this configuration works, it is not the correct one for several reasons. The first is that newer A/D boards with faster expansion buses could acquire the analog data in bursts of a few seconds per stimulus without tying up the CPU very much, thereby leaving the host CPU free for stimulus generation. The second reason is that the resource that has potentially an almost unlimited demand is the feedback and analysis. If the machine doing this component has either of the other real time tasks (analog signal acquisition and stimulus generation), then there is a very finite limit to what processing can be done on the data acquired from a particular stimulus, without creating an undue delay before the next can be generated.

This leads to another configuration. One machine does both data acquisition and stimulus generation. This is a dual monitor machine with one monitor displaying stimuli to be projected onto the retina, and a second monitor displaying control information to the experimenter. Difficult hand-shaking tasking is eliminated because all real time functions are done on one machine, an ordinary off-the-shelf Pentium. The second computer, called Data Spy, does the virtual oscilloscopes for each channel, and all the analysis and cell response display functions asynchronously, by accessing files written by the real-time machine over the local area network (LAN). Since this machine functions asynchronously by accessing files on its hard drive, different types of analysis can be selected at different times, even during a data run. If data arrives faster than it can process, it simply falls behind a few stimuli, but this has no effect on data collection. It even allows the operator to stop a given kind of analysis, and start a different one from scratch, from the first stimulus, in the middle of the data run.

Software for using the array consists of 3 main components: data (voltage) acquisition, stimulus generation synchronized with data acquisition, and output storage and user display/interface.

Array outputs are electrically connected via integral connector to the amplifier described above. Output of this amplifier is electrically connected to a storage device, such as analog to digital converter board in a Windows computer. Software contained in the system controls the state and operation of the analog to digital converter board, and synchronizes acquisition with stimulus generation (B) functions, and user display/output (C) functions. The entire system can operate on a single computer, or multiple computers. Software can control properties of the amplifier circuitry described above, including application of voltages or pulse trains to specific array elements under software program control.

Software synchronized acquisition of array from the data with presentation of graphic pictures on a windows computer monitor. Software can generate sounds, or voltage outputs to control devices such as pumps and relays, or other instrumentation.

In the present embodiment, stimulus synchronization with data acquisition uses “Direct-X” Microsoft software, but could also use other “low-level” control software. Stimuli generated include full screen graphics, changeable at every frame, at frame rates of 100 Hz or higher, with data acquisition synchronized to a specific phase of the vertical retrace pulse of the computer monitor. This allows synchronization of the data acquisition to be within one sample time (<1 millisecond at 4000 Hz sampling rate, for example) of a specific phase of the monitor vertical retrace, and therefore of the stimulus presentation.

Stimulus presentation described above include both the presentation of pre-computed image files, and the movement and merger of images by dynamic computation of graphic images at the monitor frame rate of 100 Hz or better. Examples of images synchronized with data acquisition include moving rectangular objects, moving gratings, and movement of any arbitrary bitmap, and appearance and disappearance of these objects at user specified times during the experiment.

Operation of data acquisition synchronized with stimulus generation produces output files that are initially stored in the computer RAM memory, and also, at intervals, stored on the computer hard drive, or transmitted or a network connection to another computer or other device. Output files can contain entries that consist of the voltage/current derived from or applied to any or all of the array elements, the exact analog to digital sample number or time, the status or the stimulus display, that status of any output device such as a pump or relay or other instrument controlled by the software. The files may contain header or other experimental information, and may contain user entries made before or during the data acquisition, and any results of processing the data acquired in the same or other files.

The software also generates a user interface that reports the result of data acquired from each, any or all of the array elements, the status of the elements, summary data that combines information from multiple elements, instructions to the user, graphical plots of the data acquired, comparison of the data acquired with other, previously acquired data or with mathematical models, suitable user interface for control of the array elements in voltage or current acquisition mode, or output mode, suitable user interface for control of the graphical display or stimulus or interface with other connected devices, and control of network interchange of information. Such user interface can be on the computer monitors or via audio output.

Spike extraction. At the end of an experiment, there are a set of large files, each of which contains the analog data recorded at 4 to 10 kHz per channel, for every channel of that array and for every stimulus in that data run. Next a file is generated that contains the times of occurrence of the spikes from each cell recorded, for each stimulus. Storing the analog “raw” data and then extracting spike times requires some processing, but has a number of important advantages. First, the precision of the timing of the spikes is much better when they are located in a 4 kHz analog trace, than reading a clock when the spike waveforms cross the threshold of a hardware Schmitt trigger set for the entire data run, because the actual peak of the waveform or other attribute can be located in time precisely. Second, some of the electrodes in the array are necessarily not optimally positioned to yield the largest signal to noise spikes, or have spikes from several cells, so that the ability to detect and discriminate spikes by processing the analog signal offline is vastly superior to the one shot Schmitt trigger hardware method.

An outline of the extraction method follows, which is similar to that used by Nordhausen (1996). For each channel, the first 5-10 stimuli are searched for candidate spikes, based primarily on spike amplitude. If true spikes appear to be present, even if of low signal to noise, a template building mode is entered. In this mode, the record is searched for more of these likely spike events, and they are individually selected to form an average template. Once this template is stable, a large portion of the data run for that channel is cross correlated with the template. The distribution of normalized cross correlation values is plotted, as well as the peri-stimulus time (PST) histograms as a function of the cross correlation value. True spikes will tend to cluster around a single peak in the distribution of correlation values, and will also tend to fall in the same PST bins. The PST histograms can be seen to broaden, or shift, when noise spikes, or spikes from a different cell of lower cross correlation value are included.

Once the first set of spikes are identified from a record, they are removed and the record is processed again to produce a new template. This iterative process allows the extraction of up to four different spikes from a given record. This process is repeated for each record, and a new file is created which has spike times from each cell for each channel, using the actual channel each cell was recorded for the first spike type on each channel, and pseudo-channels for multiple spikes extracted from each channel. From these files are generated typical plots such as the PST histograms, cross correlation histograms, polar plots for movement, and so forth.

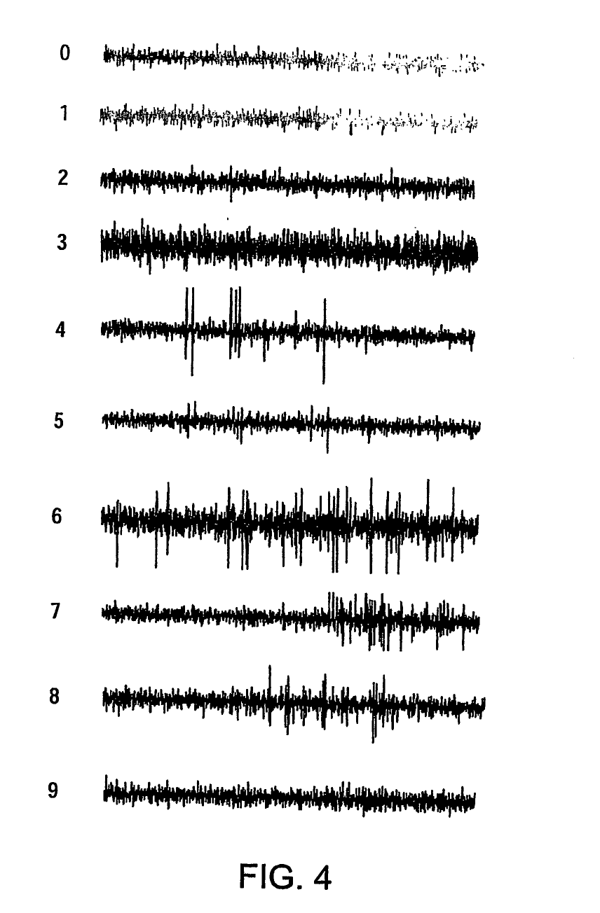

FIG. 4 shows the 10 “raw” analog data-captured traces from a 10 element array. Even at this low resolution scale, it is clear that 5-6 of the electrodes have easily discernable spikes; the spikes on some channels are small and not very evident at this scale. In the retina, virtually all the electrodes in these arrays work in that they record spikes at some retinal location or depth. At any particular depth and placement, about half of the elements will record easily discernable spikes at one time, with some electrodes recording several separable units. Thus, the total number of recordings recovered is typically on the order of the number of electrodes in the array. As expected, larger electrode tips placed in areas of the retina of high ganglion cell density, such as the visual streak, record more cells per electrode, while smaller tips in sparser regions record fewer.

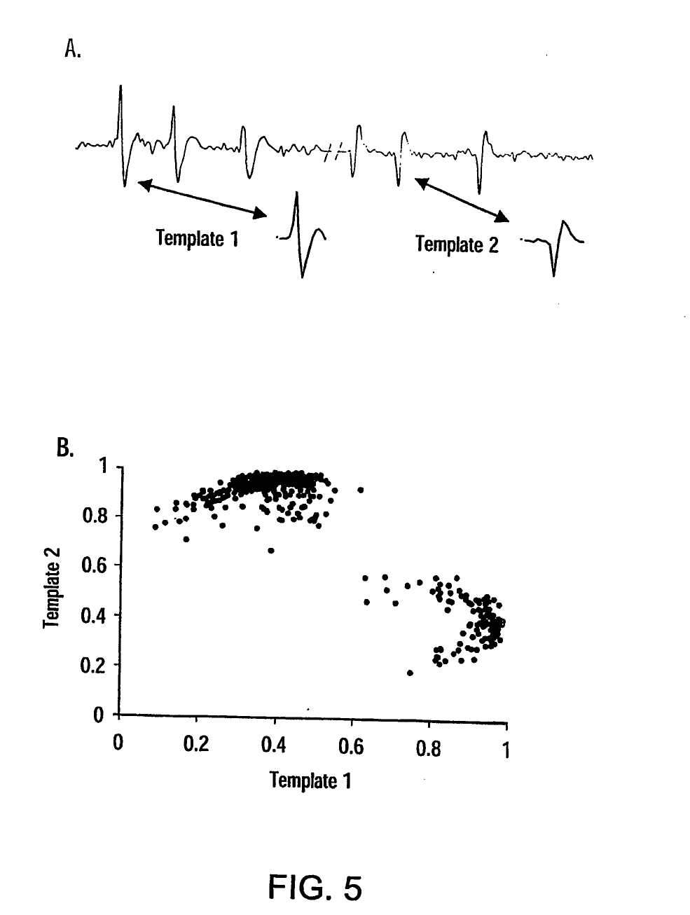

FIG. 5 shows the use of the template method for reliably extracting multiple spikes from a single microelectrode recording. The great advantage of templates is the ability to separate different spikes purely on the basis of shape, when they could not be reliably distinguished on the basis of pure amplitude. All channels are processed offline in this manner, although the system contains a hardware Schmitt trigger as typical in single microelectrode recording, and a software threshold discrimination on the virtual oscilloscopes to get real time feedback about the type of cells currently being sampled and the stability of the recordings.

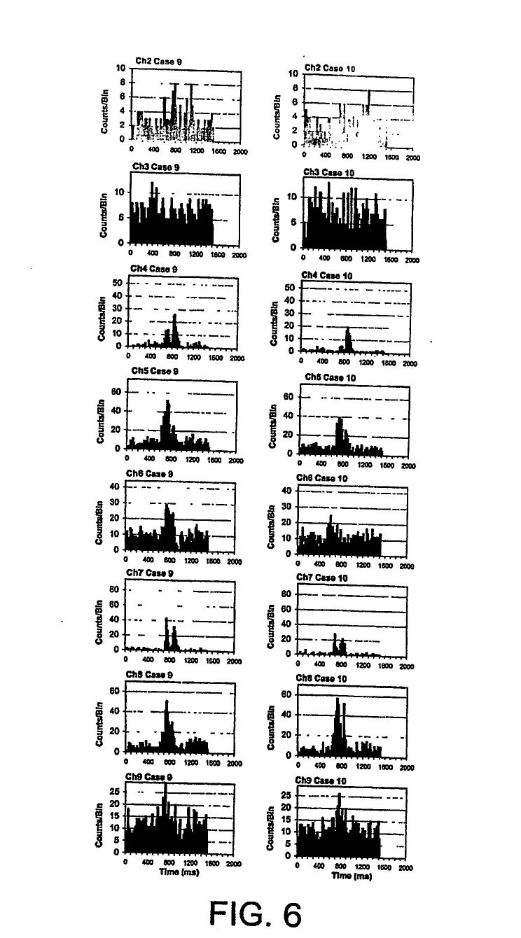

FIG. 6 shows the peri-stimulus time (PST) histograms of 8 units recovered from the traces in FIG. 4, which in turn were obtained from the 10 electrode array in FIG. 2B. Eight units were recovered. Each recovered unit appears to be from a distinct cell from the appearance of the PST histograms, although we did not directly examine the waveforms across channels. Firing cross correlation functions can be computed in which a suspiciously large peak at a single fixed delay would trigger such an examination.

Aspects of the procedure (beyond those considerations common to single electrode recording) that were essential for success were primarily using electrodes with fairly long tips that extended at least a few tens of micrometers beyond the substrate, mounting accuracy, and the ability to tilt the array in pitch and yaw to get all the tips in the same horizontal plane to match that of the tissue to be recorded. A number of configurations were tried with short tips emerging less than 10 micrometers from the substrate. In retina these resulted primarily in axon recordings (ganglion cell axons form a layer proximal to the ganglion cell soma layer). In retina such recordings are not preferred because the receptive fields of axon recordings are not confined to the region near the electrode tip, but may be found considerable distances away, and the overlap among such receptive fields was minimal.

Getting the electrode tips all in the ganglion cell layer is also critical in retinal recordings. This involved accuracy in both mounting and placement. Probably the most difficult part of the assembly procedure is the mounting. Although inter-electrode spacings of 20 micrometers or less can be achieved routinely, controlling the exact depth of all the tips with manually applied drops of the polyurethane bonding agent requires skill and patience, and the spacing is not entirely uniform in either the X-Y, or Z dimensions (as can be seen in FIG. 2B). Nevertheless, even perfect electrode-electrode depth alignments proved to be useless without the ability to align the array with the ganglion cell layer. Prior to installing the 2 tilt axes, many early arrays, some as large as 32 elements, appeared only to have a few viable channels and were needlessly discarded.

The instant invention has definite advantages compared to conventional single cell recoding. One is stability. Since the array has a distributed, but punctuated, “footprint” across the retina, there is little movement over time of the tissue with respect to the array over time. Recordings with this array are typically stable for 4-6 hours, and usually degrade gracefully even after that period. The arrays are also very robust. To generate the photomicrograph in FIG. 2B, we actually drove the array tips onto the surface of the glass graticule to get the tips and graticule in focus simultaneously, without damaging the array. Arrays can be repeatedly driven into the bottom of a plastic Petri dish, with no apparent damage because the carbon fiber tips flex, and return to their original position without breaking or taking on a permanent bend. So far, very few electrode elements have “dropped out” of function in any of our arrays over many months of use.

The array allows recording mammalian cells with a multi-electrode array system whose construction is within the reach of virtually any electrophysiology laboratory. The array is robust, and holds stable recordings for many hours, and is usable for months or longer. The near transparency of the array allows visualization of the tissue through it. This is particularly useful when recording from retina, because visual stimuli can reach the retina through the array. Transparency is also important in recording from brain slices or tissue cultures, and also has the advantage of allows lab personnel to view the underlying tissue without removing the array. The instant invention is also useful for combining array recording with optical imaging. In addition, because the recording elements of the arrays are carbon fibers, they are potentially useful for recordings other than voltage, by the use of coatings that respond to pH or the presence of any organic chemical.

The fact that these array recordings are achieved with rather mundane electronic amplification indicates that the signal-to-noise achieved with the array elements is comparable to single electrode recordings with standard microelectrodes. The individual microelectrodes in these arrays can be made in less than 10 minutes. A layer of 8-10 electrodes can be assembled in a few hours. Although most of the arrays we have used to date have had 16 or fewer elements, the only limit to this number is the patience of the array builder. Inter-element spacings of less than 20 micrometers are easily achieved.

The closest extant array to the one reported herein is that reported by Kruger and Bach (1981), which assembled individual microelectrodes into an array with a spacing of 160 micrometers, a spacing much larger than that allowed by the present invention (less than 20 micrometers). However, the array of Kruger and Bach, designed for recording in cortex, was not transparent, and thus not suitable for the retinal recording configuration we have in mind, such as stimulating through the electrode array. Kruger and Bach also used metal wire whose tips extended 2.5 mm from the substrate, a larger distance than in the present invention which uses carbon fibers. Moreover, the manufacture of our individual microelectrodes is far easier and more controllable than that in many other arrays, and the use of carbon fibers has potential applications for electrochemical detection (which we have not explored).

One particular advantage of achieving electrode spacing less than 20 micrometers is the ability to use rather small tips to over-sample an area of the tissue so that a high proportion of the cells are recorded, but on separate channels. This is, in turn, related to the problem of recovering multiple cells from individual channels when it is desired to do firing cross correlation measurements. When multiple cells are picked up on a single channel, spikes that occur at approximately the same time will necessarily have overlapping waveforms. The resultant waveform will correspond to neither of the templates of the two cells, and, in the worst case, may actually have no amplitude component above the threshold set for considering events as spikes. Because cross correlation studies (Singer et al., 1997) have most frequently shown peaks of firing coincidence near zero milliseconds delay, the problem of spike collision in multiple cell per electrode recordings is worst at the very delay of most interest. Therefore, it is clearly desirable to use more electrodes at finer spacing than the alternative.

II. Prosthetic Device

Carbon fibers are intrinsically a suitable material as electron donor and acceptors, and they therefore can be used without any coating in both receiving voltage signals from neurons and stimulating neurons with voltage signals. They can also have coatings such as used for recording to enhance either receiving or stimulating. Stimulation and receiving can be combined in the same array. Some array elements may receive voltage signals, and these recordings could be filtered by a band-pass amplifier, which would then either then other array elements would use the output of that processing to stimulate neurons to control, enhance, replace CNS function, either lost due to disease or degeneration, or to enhance normal function such as in a direct CNS interface.

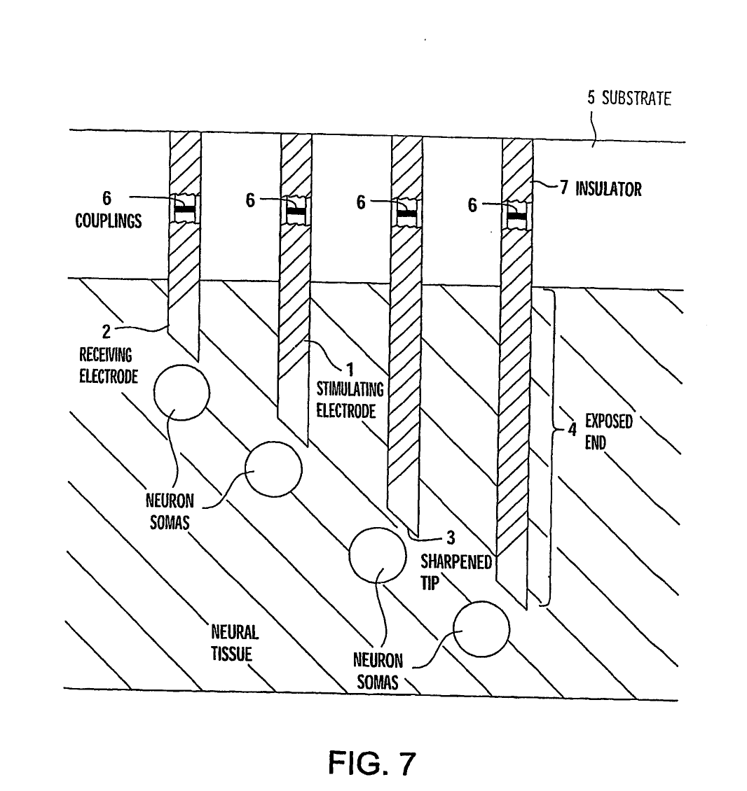

A small portion of a prosthetic device for stimulating and receiving in neural tissue with only minimal invasiveness is shown in FIG. 7. The prosthetic device described herein can be used for stimulation at millivoltage levels, as well as for receiving millivoltage signals, and can be implanted as a prosthetic device in a living body. Additionally, another embodiment of the same invention may have electrodes 1 which are for stimulating neurons, and may have other electrodes 2 for receiving voltage signals from the neural tissue, and the voltage signals received may be used to alter the activation pattern of the stimulating electrodes in the same array. The sharpened end 3 of the electrode is the most distal portion which is revealed by discontinuation of the electrical insulator. The exposed end 4 of the electrode is that portion of the electrode which is not housed within the substrate 5. The couplings of the carbon fibers and metal wires are 6. The electrical insulator covering a portion of each electrode is 7. The configuration of the array when used as an implanted prosthesis is different than is shown in FIG. 1A, 1B, and 2B. The structure of the array for use as a prosthetic device (i.e., for stimulating or recording in neural tissue, or for doing both simultaneously through electrodes in the same multielectrode device) would be different than the structure of the array used for recording in the retina. One of the difficulties of stimulating neural tissue artificially in a manner that would replicate natural stimulation is: how does one deliver enough stimuli to tissue that is multi-layered or otherwise too thick to allow stimulation only on the surface of the structure, without also damaging the underlying tissue by driving electrodes into the tissue and damaging it thereby? Another problem is that metal electrodes present the possibility of corrosion, especially when implanted for long periods of time. A number of efforts have been made to create such devices, but the present invention solves these and other problems much better than any existing device.

Spacing of individual electrodes in the array can be less than 10 micrometers between centers. The electrode spacing is a function of the width of the carbon fibers; i.e., the wider the fibers the more spacing required. The insulation layer (e.g., polyurethane) on each electrode is less than 2 microns. Thus, five micron carbon fiber electrodes, for example, could be on centers less than 10 microns apart because the only additional width required between electrodes is the insulation for each electrode. As a further example, a 12 micron carbon fiber array could have electrodes whose centers are spaced apart less than 20 microns. Choice of the width of fibers depends on the desired length of extension beyond the substrate (herein referred to as “protrusion distance”). A wider carbon fiber is used when greater protrusion distance is required.

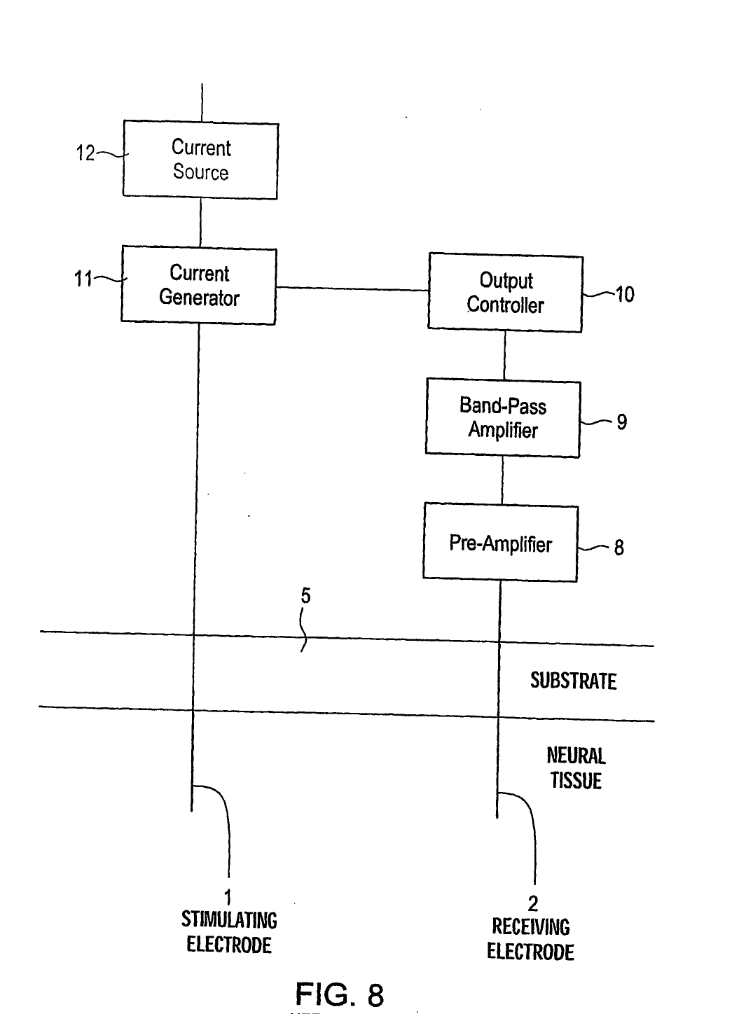

The software described above can control, by prior instruction sets, manipulation of the array within or between samples in the recording device and, in the prosthetic device, placement of voltages or currents on individual array elements in conjunction with data obtained up to the present instant. For example, for a neural prosthesis used to stimulate neural tissue such as cortex or the spine, the software could acquire neural data from any or all array elements, process the data according to a mathematical model, and output pulses on any or all array elements to stimulate neurons in the vicinity of the array, or in some other array, or control some other device. This is an advantageous feature of the array: it allows simultaneous receipt of voltage signals from neurons and stimulation of neurons with voltage signals. This has important advantages for applications in the nervous system in which feedback loops perform vitally important neural processing of information and stimulus. For example, it is well known that most axons in the LGN of the thalamus project to primary visual cortex (thalamo-cortical projections), also known as area V1. It is also known that the largest output of axons from visual cortex V1 goes to the thalamus where certain vision-related processing occurs. One theory is that these cortico-thalamic projections are important in determining attention, as salient features in cortex are somehow excited further and weaker features are inhibited in the thalamus. The unprecedented ability of the present invention to pack large numbers of electrodes into a very small area means that the receiving electrodes could serve a function similar to the cortico-thalamic feedback projections by connecting the receiving electrodes to a band-pass amplifier or other devices implanted nearby whose output would then, through coupling with the stimulating electrodes, inhibit the weaker features in the visual field but strengthen or excite the more salient. These stimulating electrodes would be analogous to thalamo-cortical projections. Thus, simultaneous stimulation and recording could allow development of visual attention, an unprecedented feature for artificial vision. This is only one example of how feedback loops could be important for neural prosthetics. FIG. 8 is a diagram of a feedback loop which could be constructed to utilize an artificial feedback mimicking that of the CNS and PNS. FIG. 8 contains a receiving electrode 2, extending through the array's substrate 5, coupled to a pre-amplifier 8, then coupled to a band-pass amplifier 9, then coupled to an output controller 10, then coupled to a current generator 11. A current source 12 is also coupled to the current generator 11.

Further, the ability to convert a stimulating electrode to an electrode for receiving voltage signals within the same multielectrode array would allow flexibility for adjusting the pattern of activation of a given area of neural tissue with feedback and direction from a human subject. This type of feedback, analogous to that of an epilepsy patient under local anesthetic during surgery to remove the affected cortical tissue, would allow a doctor to make adjustments in the invention's stimulation pattern with instructions based upon the patient's sensory perceptions. Additionally, adjustments could be made after conclusion of surgery.

Another significant aspect of the present invention is that the individual electrodes can be varied in length to target different layers or areas of neural structures so as to ensure stimulation and recording in areas much more diverse than previous devices have allowed. This variable protrusion distance on different electrodes in the same prosthetic device is a major advance over the prior art. For instance, neo-cortex in most mammals is 2-4 millimeters thick and has identifiable layers, and stimulating and recording in different layers would be possible with the current invention by varying the lengths of the multiple protrusion distances of the individual electrodes. The substrate from which the individual electrodes protrude would rest against the surface of the neural structure and the individual electrodes would pierce the surface to the desired depths. Because individual electrodes can be less than 10 microns apart (depending on the thickness of the carbon fibers), these electrodes can slide through the tissue to deeper layers or thicknesses with very minimal damage to the neurons in the upper layers. This aspect of the invention is depicted in FIG. 7.

Individual electrodes of different protrusion distances are insulated to a position in the neighborhood of the etched point (as described above) so that the sharpened point is the only portion of the protrusion distance un-insulated. This allows delivery of charge to a precise location. For example, assuming a protrusion distance of 2 mm, the current would travel only to tissue at the exposed sharpened and un-insulated tip which is less than 15 microns.

For electrophysiology, the software can specifically display the output of each, any, or all array elements as a result of the stimulus to form a “map” of the response versus stimulus parameter such as position, intensity or other parameter. The software can display post stimulus time histograms of the firing of action potentials of neurons recorded by the array. The software can display, following analysis by the software, of cross channel data features such as synchronous firing of neurons, or detection of particular firing patterns in either single or multiple channels. The software can allow interactive setting of thresholds or other parameters or online analysis of data acquired, and display during the data acquisition, results from all data acquired prior to the present instant.

III. Bio-Sensing Device

The carbon fiber array may also be used as a sensor for organic compounds, and this sensing can be done separately or simultaneously (in the same array) with electrophysiological recording or stimulation. The array can be implanted or placed temporarily in vivo with coatings or releasable substances which enhance tissue compatibility or neural interface, such as neural growth factors, cell adhesion molecules, or even stem cells.

Carbon fibers act as electron donor or acceptors and so can participate in reduction—oxidation detection of oxidizable or reducible biological substances such as neurotransmitters like dopamine, by application of a voltage to the fiber, and monitoring of current flow in the presence of the biological substrate. Coatings can be applied to the fiber tips for more specific detection. The array is mounted on a standard connector, and therefore can be disposable. Multiple sensing elements can be placed in a very small space, such as a single droplet of fluid, capillary bed, or any biological substance. Multiple voltages can be applied to different array elements, either simultaneously, or sequentially, to enhance the ability to detect and identify a particular biochemical species. Different coatings can be used on different array elements either to (1) enhance the specificity of detection and identification of a chemical species, or (2) allow simultaneous detection of multiple biochemical or chemical substances. For example, by means of an amperometry device, the array could make physiological measurements relevant to blood pressure (any combination of neural activity and bio-sensing) and produce an output to control other neurons affecting blood pressure, or activate a pump that released a substance that affected blood pressure. Amperometry involves applying a fixed or pulsatile voltage between 2 electrodes, and determining the current passing in the circuit between the electrodes. Depending on the electrodes, the current is related to the concentration of chemical species that are detectable by the electrodes at the voltage potential. Amperometry devices are produced, for example, by Abtech Scientific, Inc. of Richmond, Va.

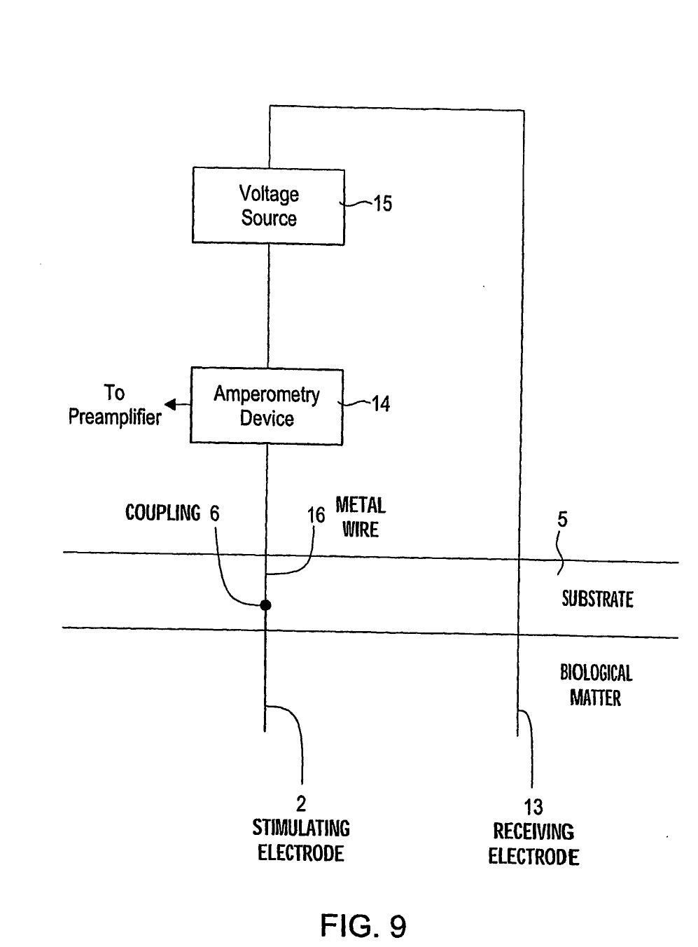

FIG. 9 is a flow chart showing the circuit for the biosensing electrode, with a receiving electrode 2 extending beyond the substrate to a coupling 6 with a metal wire 16, which is coupled to an amperometry device 14, which is coupled to a voltage source, which is coupled to a reference wire 13, which also extends beyond the substrate 5 and into the biological material being sampled.

### REFERENCES

Amthor F R and Oyster C W. Spatial organization of retinal information about movement detection. Proc. Natl. Acad. Sci. USA., 1995; 92(9): 4002-4005.

Amthor, F R, Tootle, J S, Yildirim, Abidin, A new transparent multi-unit recording array system fabricated by in-house laboratory technology. J. Neurosci. Methods, 2003; 126(2): 209-219

Grumet A E, Wyatt, Jr. J L and Rizzo, III J F Multi-electrode stimulation and recording in the isolated retina. J. Neurosci. Methods, 2000; 101: 31-42.

Heuschkel M O, Fejtl M, Raggenbass M, Bertrand D and Renaud P. A three dimensional multi-electrode array for multi-site stimulation and recording in brain slices. J. Neurosci. Methods, 2002; 114: 135-148.

Meister M, Pine J and Baylor D A. Multi-neuronal signals from the retina: acquisition and analysis. J. Neurosci. Methods, 1994; 51: 95-106.

Nordhausen, C. T., Maynard, E. M., and Normann, R. A. Single Unit Recording Capabilities of a 100 Microelectrode Array. Brain Res., 1996; 726:129-140.

Sandison M, Curtis A S G and Wilkinson C D W. Effective extra-cellular recording from vertebrate neurons in culture using a new type of micro-electrode array., J. Neurosci. Methods, 2002; 114: 63-71.

Singer W, Engel A K, Kreiter A K, Monk, M H J, Neuenschwander S and Roelfsema P. Neuronal assemblies: necessity, signature and detectability. TINS, 1997; 1(7):252-261.

Thorpe S, Delorme A and Van Rullen, R. Spike-based strategies for rapid processing. Neural Networks, 2001; 14:715-725.

1. A multielectrode array for receiving voltage signals from neurons, the multielectrode array comprising:
A substrate;
At least two electrodes partially contained in said substrate, each of said at least two electrodes having a carbon fiber with an exposed end extending beyond the substrate, an unexposed end within the substrate, and a center, said exposed end capable of being embedded in neural tissue, and
An electrical insulator covering at least a portion of each of the at least two electrodes.

2. The multielectrode array as in claim 1, where each of said at least two carbon fibers has any combination of at least one of the characteristics independently selected from the group consisting of: a coating on the exposed end, a sharpened tip, and varied length of the exposed end.

3-10. (canceled)

11. A multielectrode array for receiving voltage signals from neurons, the multielectrode array comprising:
A substrate;
At least two electrodes partially contained in said substrate, each of said at least two electrodes having a biocompatible wire with an exposed end extending beyond the substrate, an unexposed end within the substrate, and a center, said exposed end capable of being embedded in neural tissue, and said at least two electrodes being spaced from one another so that the spacing between the centers of said exposed ends of each said biocompatible wire does not exceed 45 microns; and
An electrical insulator covering at least a portion of each of the at least two electrodes.

12-24. (canceled)

25. A multielectrode array for receiving voltage from neurons and stimulating neurons with voltage signals, the array comprising:
A substrate;
At least one receiving electrode partially contained in said substrate, each said at least one receiving electrode having a carbon fiber with an exposed end extending beyond the substrate, an unexposed end within the substrate, and a center, said exposed end capable of being embedded in neural tissue, said carbon fiber's unexposed end coupled to a metal wire's first end, said metal wire's second end being coupled to the input to a pre-amplifier;
At least one stimulating electrode partially contained in said substrate, each said at least one stimulating electrode having a carbon fiber with an exposed end extending beyond the substrate, an unexposed end within the substrate, and a center, said exposed end capable of being embedded in neural tissue, said carbon fiber's unexposed end coupled to a metal wire's first end, said metal wire's second end being coupled to the input to a current generator; and
An electrical insulator covering at least a portion of the said at least one receiving electrode and the said at least one stimulating electrode.

26. The multielectrode array as in claim 25, where each of said at least two carbon fibers has any combination of at least one of the characteristics independently selected from the group consisting of: a coating on the exposed end, a sharpened tip, and varied length of the exposed end.

27-30. (canceled)

31. A multielectrode array for stimulating neurons with voltage signals and for receiving voltage signals from neurons, the multielectrode array comprising:
A substrate;
At least one stimulating electrode partially contained in said substrate, each at least one stimulating electrode having a biocompatible wire with an exposed end extending beyond the substrate, an unexposed end within the substrate, and a center, said exposed end capable of being embedded in neural tissue and said unexposed end coupled to the input to a current generator;
At least one receiving electrode partially contained in said substrate, each at least one receiving electrode having a biocompatible wire with an exposed end extending beyond the substrate, an unexposed end within the substrate, and a center, said exposed end capable of being embedded in neural tissue and said unexposed end coupled to the input to a pre-amplifier; and
An electrical insulator covering at least a portion of the said at least one receiving electrode and the said at least one stimulating electrode.

32. The multielectrode array as in claim 31, where each of said biocompatible wires has any combination of at least one of the characteristics independently selected from the group consisting of: a coating on the exposed end, a sharpened tip, and varied length of the exposed end.

33-36. (canceled)

37. A multielectrode array for receiving voltage from neurons, stimulating neurons with voltage signals, and for providing feed-back between neurons, the array comprising:
Said substrate;
At least one receiving electrode partially contained in said substrate, each said at least one receiving electrode having a carbon fiber with an exposed end extending beyond the substrate, an unexposed end within the substrate, and a center, said exposed end capable of being embedded in neural tissue, said carbon fiber's unexposed end coupled to a metal wire's first end, said metal wire's second end being coupled to the input to a pre-amplifier;
At least one stimulating electrode partially contained in said substrate, each said at least one stimulating electrode having a carbon fiber with an exposed end extending beyond the substrate, an unexposed end within the substrate, and a center, said exposed end capable of being embedded in neural tissue, said carbon fiber's unexposed end coupled to a metal wire's first end, said metal wire's second end being coupled to the input to a current generator;
At least one feedback loop constituting a system partially contained in said substrate, containing a first carbon fiber with an exposed end capable of being embedded in neural-tissue, said first carbon fiber's unexposed end coupled to a first metal wire's first end, said first metal wire's second end coupled to the input to a pre-amplifier, the output of said pre-amplifier coupled to the input to a band-pass amplifier, the output from said band-pass amplifier coupled to the input to an output limiter, the output of said output limiter coupled to a current generator, said current generator coupled to the said second end of a second metal wire and also to a current supply, the said first end of said second metal wire coupled to the unexposed end of a second carbon fiber; the said exposed end of said second carbon fiber capable of being embedded in neural tissue; and
Said electrical insulator for each of the said at least one receiving electrode, said at least one stimulating electrode, and said at least one feedback loop.

38. The multielectrode array as in claim 37, where each of said at least two carbon fibers has any combination of at least one of the characteristics independently selected from the group consisting of: a coating on the exposed end, a sharpened tip, and varied length of the exposed end.

39. The multielectrode array as in claim 37, where the output limiter is deleted.

40-45. (canceled)

46. A multielectrode array for stimulating neurons with voltage signals and for receiving voltage signals from neurons, and for providing feed-back between neurons, the multielectrode array comprising:
A substrate;
At least one stimulating electrode partially contained in said substrate, each at least one stimulating electrode having a biocompatible wire with an exposed end extending beyond the substrate, an unexposed end within the substrate, and a center, said exposed end capable of being embedded in neural tissue and said unexposed end coupled to the input to a current generator;
At least one receiving electrode partially contained in said substrate, each at least one receiving electrode having a biocompatible wire with an exposed end extending beyond the substrate, an unexposed end within the substrate, and a center, said exposed end capable of being embedded in neural tissue and said unexposed end coupled to the input to a pre-amplifier;
At least one feedback loop constituting a system partially contained in said substrate, containing a first biocompatible wire with an exposed end extending beyond the substrate, an unexposed end within the substrate, and a center, said first biocompatible wire's unexposed end coupled to the input to a pre-amplifier, the output of said pre-amplifier coupled to the input to a band-pass amplifier, the output from said band-pass amplifier coupled to the input to an output limiter, the output of said output limiter coupled to a current generator, said current generator coupled to the second end of a second biocompatible wire and also to a current supply, the said first end of said second biocompatible wire capable of being embedded in neural tissue; and
Said electrical insulator for each of the said at least one receiving electrode, said at least one stimulating electrode, and said at least one feedback loop; and
An electrical insulator covering at least a portion of the said at least one receiving electrode and the said at least one stimulating electrode.

47. The multielectrode array as in claim 46, where each of said biocompatible wires has any combination of at least one of the characteristics independently selected from the group consisting of: a coating on the exposed end, a sharpened tip, and varied length of the exposed end.

48. The multielectrode array as in claim 46, where the output limiter is deleted.

49-54. (canceled)

55. A biosensing device, comprising:
A substrate;
At least two biosensing electrode circuits, each said at least two biosensing electrode circuit containing a biosensing electrode having a carbon fiber with an exposed end extending beyond the substrate, an exposed end within the substrate, and a center, said carbon fiber being coupled with the first end of a metal wire, said metal wire's second end-being coupled with an amperometry device, said amperometry device being coupled with a voltage source and a pre-amplifier, said voltage source being coupled with a reference wire; and
An electrical insulator covering at least a portion of each of the biosensing electrodes and the reference wire.

56. The biosensing device as in claim 55, where each of said at least two carbon fibers has any combination of at least one of the characteristics independently selected from the group consisting of: a coating on the exposed end, a sharpened tip, and varied length of the exposed end.

57-60. (canceled)

61. A biosensing device, comprising:
A substrate;
At least two biosensing electrode circuits, each of said at least two biosensing electrode circuits containing a biosensing electrode having a biocompatible wire with an exposed end extending beyond the substrate, an exposed end within the substrate, and a center, said biocompatible wire being coupled with an amperometry device, said amperometry device being coupled with a voltage source and a pre-amplifier, said voltage source being coupled with a reference wire, and said at least two biosensing electrodes being spaced from one another so that the spacing between the centers of said exposed ends of each said carbon fiber does not exceed 20 microns; and
An electrical insulator covering at least a portion of each of the biosensing electrodes and the reference wire.

62. The biosensing device as in claim 61, where said biocompatible wire has any combination of at least one of the characteristics independently selected from the group consisting of: a coating on the exposed end, a sharpened tip, and varied length of the exposed end.

63-65. (canceled)

66. The multielectrode array as in claim 1, wherein the at least two electrodes are selected from the following: receiving electrodes and stimulating electrodes.

67. The multielectrode array as in claim 1, wherein said at least two electrodes being spaced from one another so that the spacing between the centers of said exposed ends of each said carbon fiber does not exceed 20 microns.

68. The multielectrode array as in claim 1, wherein said at least two receiving electrodes being spaced from one another so that the spacing between the centers of said exposed ends of each said carbon fiber does not exceed 45 microns.
